(19) United States
(12) Patent Application Publication
(10) Pub. No.: US 2024/0285212 A1
(43) Pub. Date: Aug. 29, 2024

(54) TECHNIQUE FOR HOLDING AN ELECTRODE

(71) Applicant: CUMULUS NEUROSCIENCE LIMITED, Belfast (GB)

(72) Inventors: Bryan Murphy, Dublin (IE); Ian McCullough, Comber (GB); Glyn Griffiths, Dublin (IE); Terence Kealy, Kilkenny (IE); Sara Urasini, Bray (IE); Martin McNeill, Ballymena (GB)

(21) Appl. No.: 18/573,347

(22) PCT Filed: Jun. 22, 2021
(86) PCT No.: PCT/EP2021/066998

§ 371 Date: Dec. 21, 2023

(51) Int. Cl. A61B 5/291; A61B 5/256; A61B 5/282

(52) U.S. Cl. CPC A61B 5/291; A61B 5/256; A61B 5/282; A61B 2562/227

(57) ABSTRACT

Disclosed is an electrode assembly, comprising an electrode configured to be placed on a surface of a subject's body for recording an electric body signal, an electrode receptacle configured to receive the electrode, and a coupling mechanism configured to couple the electrode to the electrode receptacle, the coupling mechanism having a locked configuration, in which the electrode is coupled to the electrode receptacle, and an unlocked configuration, in which the electrode is decoupled from the electrode receptacle. The disclosure also relates to an electrode, an electrode receptacle and a system.

### TECHNICAL FIELD

The present disclosure generally relates to an electrode assembly comprising an electrode and an electrode receptacle. The disclosure also relates to an electrode, an electrode receptacle and a system.

### BACKGROUND

Bioelectric measurements such as electroencephalography, EEG, or electrocardiography, ECG, rely on electrodes being placed on a surface of a subject's (e.g., an animal's or a human's) body for recording electric signals. For instance, EEG caps comprising a plurality of so-called wet electrodes are known in the art. Such electrodes are usually permanently fixed to the EEG cap and can sense EEG signals when contacting the body surface via a conductive gel.

Depending on the measurement scenario (e.g., depending on the subject, depending on the measuring environment, etc.), a different type of electrode (e.g., a larger electrode, a differently shaped electrode etc.) may yield more accurate measurements. With the permanently fixed electrodes of known EEG caps, this means that for each different type of electrode, a different EEG cap would be needed.

In case one or more of the electrodes of the EEG cap fail, restoring the functionality of the EEG cap may be cumbersome. For example, replacement of the failed, permanently fixed electrodes may be difficult or even impossible without damaging the EEG cap. In many cases, replacing an electrode requires soldering a new electrode onto existing wiring of the EEG cap. Such soldering is not only difficult, but may even lead to faulty electrical connections or unpredictable electrical properties of the solder connection, thereby potentially negatively impacting EEG measurements.

In addition, the cleaning of permanently fixed electrodes may be difficult. EEG caps including multiple electrodes may require regular cleaning, for instance after each use. This may be cumbersome if the entire device needs to be cleaned, in particular if the cap itself comprises soft materials that soak up solvents used for the cleaning. Such caps having soaked up solvents may be inconvenient for the user.

Similar issues may persist with existing ECG measurement devices comprising a plurality of ECG electrodes.

### SUMMARY

There is a need for a technique that solves one or more of the aforementioned or other problems.

According to a first aspect, an electrode assembly is provided. The electrode assembly comprises an electrode configured to be placed on a surface of a subject's body for recording an electric body signal, an electrode receptacle configured to receive the electrode; and a coupling mechanism configured to couple the electrode to the electrode receptacle, the coupling mechanism having a locked configuration, in which the electrode is coupled to the electrode receptacle, and an unlocked configuration, in which the electrode is decoupled from the electrode receptacle.

The electric body signal may be a bioelectric signal such as an electroencephalographic, EEG, signal or an electrocardiographic, ECG, signal. The electrode receptacle may be configured to receive the electrode by at least partially enclosing the electrode. The electrode receptacle may be or form a housing for at least a part of the electrode. The electrode receptacle may be generally pot-shaped. The coupling mechanism may be configured to couple the electrode to the electrode receptacle by holding the electrode in a fixed spatial relationship relative to the electrode receptacle. The coupling mechanism may be configured to couple the electrode to the electrode receptacle by removably attaching the electrode to the electrode receptacle.

The coupling mechanism may comprise at least one of a bayonet mount assembly, a bayonet connector assembly, a turn-lock assembly, a screw assembly, a snap-fit assembly, a friction assembly, and a magnetic assembly.

The coupling mechanism may be configured to be shifted between the unlocked configuration and the locked configuration (e.g., from the unlocked configuration to the locked configuration and/or from the locked configuration to the unlocked configuration) manually. The coupling mechanism may be configured to be shifted between the unlocked configuration and the locked configuration by (e.g., only and/or manually) turning or rotating the electrode relative to the electrode receptacle. Alternatively or additionally, the coupling mechanism may be configured to be shifted between the unlocked configuration and the locked configuration by (e.g., only and/or manually) translationally (e.g., axially) moving the electrode relative to the electrode receptacle.

The coupling mechanism may comprise a male coupling feature and a female coupling feature. The electrode receptacle may comprise the male coupling feature and the electrode may comprise the female coupling feature. Alternatively, the electrode may comprise the male coupling feature and the electrode receptacle may comprise the female coupling feature. The male coupling feature may comprise or be a protrusion, a pin or a lip. The female coupling feature may comprise or be a recess, a hole, a cavity or a notch.

The coupling mechanism may comprise at least one protrusion (e.g., as the male coupling feature), wherein at least one of the electrode and the electrode receptacle may comprise a base portion having a (e.g., outer and/or inner) lateral surface. The at least one protrusion may extend (e.g., radially and/or essentially orthogonally) from the lateral surface. The coupling mechanism may comprise at least one recess (e.g., as the female coupling feature) at the respective other of the electrode or electrode receptacle, the at least one recess being configured to engage the at least one protrusion.

The at least one protrusion may be a pin or a lip. The at least one protrusion may be a radial pin of a bayonet mount. The base portion may be rotationally symmetric with respect to a first axis. The first axis may be a center axis, a rotation axis or a longitudinal axis of the base portion. The base portion may for example have a conical or cylindrical shape. For example, the base portion may be shaped essentially like a (e.g., right circular) cone or a (e.g., right circular) cylinder. The at least one protrusion may extend radially inwards from the (e.g., outer) lateral surface or radially outwards from the (e.g., inner) lateral surface. The at least one protrusion may extend essentially perpendicular to the lateral surface and/or the first axis.

In a first variant of the first aspect, the electrode comprises the base portion having the lateral surface from which the at least one protrusion extends (e.g., radially outwards or radially inwards). At least one protrusion may extend across at least (e.g., only) a part of a circumference of the lateral surface. In this variant, the at least one recess may be formed at the electrode receptacle. For example, the at least one recess is formed in or by the electrode receptacle.

In a second variant of the first aspect, the electrode receptacle comprises the base portion having the lateral surface from which the at least one protrusion extends (e.g., radially outwards or radially inwards). At least one protrusion may extend across at least (e.g., only) a part of a circumference of the lateral surface. In this variant, the at least one recess may be formed at the electrode.

The electrode receptacle may comprise an opening for receiving the electrode, the opening having an inner surface (e.g., the inner lateral surface). The electrode receptacle may comprise at least one second protrusion extending radially inwards from the inner surface. The at least one recess may be (e.g., at least or only) axially confined by the at least one second protrusion.

The opening may be rotationally symmetric with respect to a second axis. The second axis may be a center axis, a rotation axis or a longitudinal axis of the electrode receptacle or the electrode (e.g., in case the coupling mechanism is in the locked configuration). The term “axially” used herein may refer to the first axis and/or the second axis. The opening may for example have a conical or cylindrical shape. For example, the opening may be formed essentially as a (e.g., right circular) cone or a (e.g., right circular) cylinder. The at least one recess may be axially confined by the at least one second protrusion with respect to at least one of the first and the second axis. The at least one second protrusion may border or delimit the at least one recess, for example at least axially.

The at least one recess may be formed or defined by the at least one second protrusion and an inner wall of the electrode receptacle. The at least one recess may be (e.g., axially and, optionally, radially) confined by the at least one second protrusion and the inner wall. For example, an outline of the opening and the at least one second protrusion at least partially overlaps an outline of the base portion of the electrode and the at least one protrusion, when the coupling mechanism is in the locked configuration. The inner surface of the electrode receptacle may be radially distant from an outermost surface of the electrode (e.g., an outer surface of the at least one protrusion). A radial outer surface of the at least one protrusion may be (e.g., outwards) radially distant from a radial inner surface of the at least one second protrusion. In other words, the radial outer surface of the at least one second protrusion may be closer to the first and/or second axis than the radial outer surface of the at least one protrusion (e.g., when the coupling mechanism is in the locked configuration).

The base portion of the electrode or the electrode receptacle may have an axial (e.g., end) surface facing a surface of the respective other of the electrode or electrode receptacle, wherein said axial surface comprises at least one groove, wherein the at least one groove has a varying depth along its length, and wherein the at least one groove is configured to couple with at least one pin arranged at (e.g., arranged on, attached to and/or extending from) the surface of the respective other of the electrode or the electrode receptacle.

The at least one pin may be arranged such that it extends into the at least one groove when the coupling mechanism is shifted between the locked configuration and the unlocked configuration (e.g., from the locked configuration to the unlocked configuration and/or from the unlocked configuration to the locked configuration). The at least one pin and the at least one groove may be arranged such that, when the coupling mechanism is shifted between the locked configuration and the unlocked configuration, the at least one pin is moved (e.g., inside and) along the at least one groove. The at least one groove may be configured as a guide for the at least one pin.

The base portion of the electrode or the electrode receptacle may have an axial (e.g., end) surface facing a surface of the respective other of the electrode or electrode receptacle, wherein said axial surface comprises at least one blocking cavity, wherein the at least one blocking cavity is configured to couple with (e.g., the) at least one pin arranged at (e.g., arranged on, attached to and/or extending from) the surface of the respective other of the electrode or the electrode receptacle.

The axial surface may be a base surface, an axial end surface or a proximal surface of the base portion. The surface of the respective other of the electrode or electrode receptacle may be a base surface, an axial end surface or a proximal surface of the (e.g., base portion of the) respective other of the electrode or electrode receptacle. The at least one pin may extend essentially perpendicular to or axially from the surface of the (e.g., base portion of the) respective other of the electrode or the electrode receptacle. The at least one groove may extend parallel to a lateral surface of the base portion of the electrode or the electrode receptacle. The at least one groove may extend along a segment of a circle. The at least one groove may have a depth changing linearly along the length of the groove. The at least one groove may have a depth ascending along the length of the groove. The at least one groove may have an (e.g., axial) bottom surface shaped like a ramp. The at least one blocking cavity may be formed at a position distant from a radial center and/or a center of rotation of the axial end surface.

The at least one pin and the at least one blocking cavity may be arranged such that the at least one pin extends into the at least one blocking cavity when the coupling mechanism is in the locked configuration, and does not extend into the at least one blocking cavity when the coupling mechanism is in the unlocked configuration.

The at least one blocking cavity may be arranged at a longitudinal end of the at least one groove, wherein the at least one pin, the at least one groove and the at least one blocking cavity are arranged such that, when the coupling mechanism is shifted from the unlocked configuration to the locked configuration, the at least one pin is moved (e.g., inside and) along the at least one groove, and once the coupling mechanism reaches the locked configuration, the at least one pin extends into (e.g., and thereby couples with or blocks) the at least one blocking cavity.

In one embodiment, the electrode receptacle may comprise an upper housing and a lower housing, wherein the upper housing and the lower housing are configured to be coupled to one another to form a (e.g., the) housing for receiving the electrode, and wherein (e.g., the) at least one male coupling feature or (e.g., the) female coupling feature is formed by the coupled upper housing and lower housing. The upper housing and the lower housing may be coupled to one another via a snap-fit or a click-on coupling. The upper housing and the lower housing may be permanently coupled (e.g., attached) to one another. The base portion of the electrode receptacle may form the lower housing and/or the upper housing. The coupling mechanism may be configured to be shifted between the unlocked configuration and the locked configuration (e.g., only) by assembling or disassembling (e.g., coupling or de-coupling) the upper housing and the lower housing.

Optionally, the upper housing and the lower housing may be configured to hold the electrode in place (e.g., in a fixed spatial relationship relative to the electrode receptacle) when being assembled (e.g., when being in an assembled state) and may be configured to release the electrode when being disassembled (e.g., when being in a disassembled state). For instance, in this configuration, the electrode may be exclusively held within the electrode assembly by at least one protrusion of the lower housing covering the electrode. In this configuration, the electrode may only be released if the lower housing is disassembled from the upper housing.

In an alternative embodiment, the electrode receptacle may be formed as a unitary (e.g., solid or single-piece) structure, wherein all the elements described herein for the upper and lower housing are provided by the unitary structure. In this case, it may be considered that the upper housing is permanently fixed to or integrally formed with the lower housing.

The lower housing may comprise the at least one second protrusion. The upper housing may be configured to at least partially cover the (e.g., axial) opening of the lower housing (e.g., from an axial direction). The upper housing may form a base surface of an overall pot-shape of the electrode receptacle. The at least one recess may be formed by (e.g., axially and/or radially confined by, defined by or bordered by) the at least one second protrusion and the inner wall (e.g., the base surface of the overall pot-shape) of the upper housing. For example, a distance between the at least one second protrusion and the inner wall of the upper housing may match an axial height of the at least one protrusion.

The electrode or the base portion of the electrode may have an (e.g., the) axial surface facing a (e.g., the) surface of the electrode receptacle, wherein said axial surface comprises at least one trench configured to receive electronic wiring or an electrode receptacle contact. Alternatively or additionally, said axial surface may comprise an electrode contact region configured to contact an electrode receptacle contact.

The electrode assembly may further comprise an electrical pressure contact element arranged in a fixed pose relative to the electrode receptacle (e.g., attached to the electrode receptacle) and configured to contact the electrode when the coupling mechanism is in the locked configuration. Optionally, the electrode assembly may further comprise an electronic circuit board arranged on (e.g., attached to) the electrode receptacle, wherein the electrical pressure contact element is arranged on (e.g., attached and/or soldered to) the electronic circuit board. The electrode receptacle contact may be the electrical pressure contact element.

The electrical pressure contact element may be configured to exert a pressure or contact force onto the electrode when the coupling mechanism is in the locked configuration. The electrical pressure contact element may comprise a spring contact. The electrical pressure contact element and/or the electronic circuit board may be arranged on the inner wall of the upper housing and/or in a space enclosed by the upper housing and the lower housing. The electrical pressure contact element and/or the electronic circuit board may be arranged between the upper housing and the electrode when the coupling mechanism is in the locked configuration. The electronic circuit board may be a printed circuit board, PCB.

The electrode assembly may further comprise a wire electrically connected to the electrical pressure contact element and/or the electronic circuit board. The upper housing may comprise at least one through-hole, wherein at least one of the electrical pressure contact element, the electronic circuit board and the wire extends through the through-hole.

The electrode may comprise a plurality of electrode pins or electrode fingers configured to be placed on a (e.g., surface of a) subject's body. The electrode pins or fingers may be deformable, elastic or flexible (e.g., have a shore hardness between A35 and A100 according to the ASTM D2240-00 testing standard). The electrode may be a dry electrode or a wet electrode. Preferred is the use of a dry electrode. The electrode may be a molded element, for example an injection-molded element. The electrode may be made of a homogeneous material. The electrode may be made from an electrically conductive (e.g., thermo-)plastic material, for example a polymer (e.g., doped with silver or silver chloride). The electrode, or parts of it such as the pins or fingers, may be elastic, deformable or flexible.

According to a second aspect, an electrode for recording an electric body signal is provided. The electrode comprises a base portion, a plurality of electrode pins attached to the base portion and configured to be placed on a surface of a subject's body for recording an electric body signal, and at least one male coupling feature or female coupling feature for coupling to an electrode receptacle, wherein the at least one male coupling feature or female coupling feature extends radially (e.g., radially outwards) with respect to the base portion, and wherein the at least one male coupling feature or female coupling feature extends across at least (e.g., only) a part of a circumference of the base portion. The electrode of the second aspect may be part of the electrode assembly according to the first aspect. The electrode of the second aspect may additionally comprise one or more of the features described for the electrode of the electrode assembly according to the first aspect.

According to a third aspect, an electrode receptacle for an electrode for recording an electric body signal is provided. The electrode receptacle comprises an opening for receiving the electrode, wherein the electrode is configured to be placed on a surface of a subject's body for recording an electric body signal, at least one male coupling feature or female coupling feature for coupling to the electrode, wherein the at least one male coupling feature or female coupling feature extends radially (e.g., radially inwards) with respect to the opening and extends across at least (e.g., only) a part of a circumference of the opening, and an electrical pressure contact element arranged in a fixed position relative to the electrode receptacle and configured to contact the electrode when the electrode is held in a fixed position relative to the receptacle by the at least one male coupling feature or female coupling feature. The electrode receptacle of the third aspect may be part of the electrode assembly according to the first aspect. The electrode receptacle of the third aspect may additionally comprise one or more of the features described for the electrode receptacle of the electrode assembly according to the first aspect.

According to a fourth aspect, a system is provided. The system comprises the assembly according to the first aspect or the electrode receptacle according to the third aspect, and a holder configured to hold the receptacle in a fixed position relative to the surface of a (e.g., the) subject's body.

The holder may be a cap, a headband or a headset. The holder may be configured to hold the receptacle in a fixed position relative to a head of a human, for example at one of the positions of the 10-10 or 10-20 EEG electrode placement scheme.

The system may further comprise a first circuit board arranged at an element of the holder to be placed on a rear side of the user's head. The system may further comprise wiring electrically connecting the first circuit board with the electrical pressure contact element described herein. The wiring may comprise one or more (e.g., shielded) wires.

The system may further comprise a second circuit board (e.g., printed circuit board, PCB) comprising circuitry for collecting an electroencephalography, EEG, measurement signal from the first circuit board. The second circuit board may be arranged at the element of the holder to be placed on the rear side of the user's head. The second circuit board may be removably coupled to the first circuit board.

### BRIEF DESCRIPTION OF THE DRAWINGS

Further details, advantages and aspects of the present disclosure will become apparent from the following embodiments taken in conjunction with the drawings, wherein:

FIG. 1 shows a first exploded view of a first embodiment of an electrode assembly in accordance with the present disclosure;

FIG. 2 shows a second exploded view of the embodiment of FIG. 1; and

FIG. 3 shows a system in accordance with the present disclosure.

### DETAILED DESCRIPTION

In the following description, exemplary embodiments of an electrode assembly, an electrode, an electrode receptacle and a system will be explained with reference to the drawings. The same reference numerals will be used to denote the same or similar structural features.

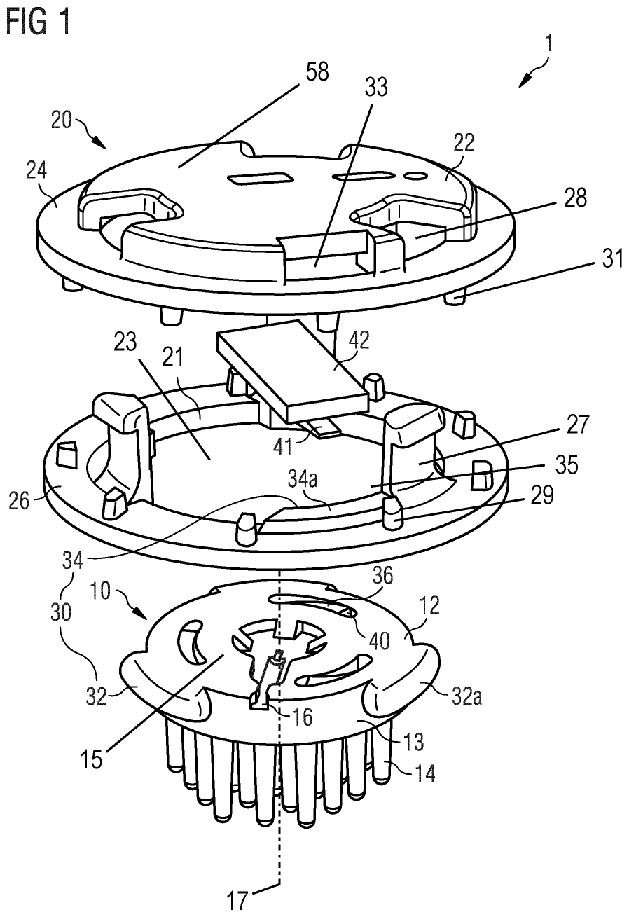

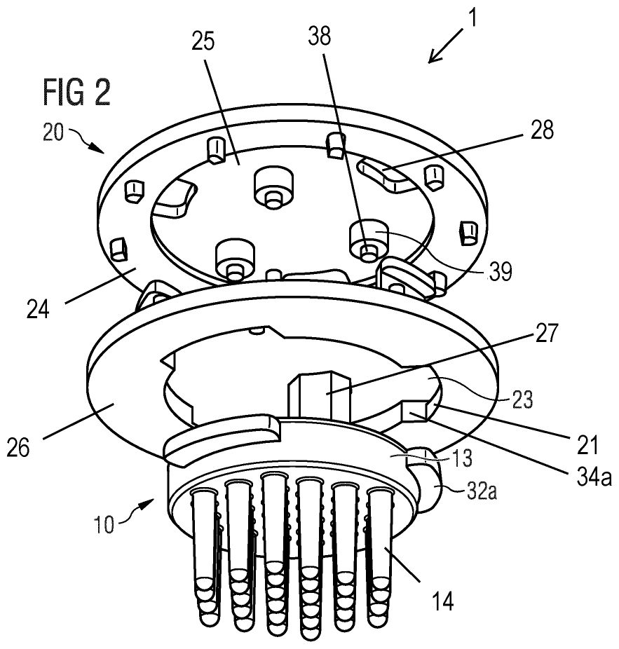

A first embodiment in accordance with the present disclosure is shown in FIGS. 1 and 2. As can be seen, an electrode assembly 1 is provided comprising an electrode 10 and an electrode receptacle 20 configured to receive the electrode 10.

The electrode 10 may comprise an essentially cylindrical base portion 13. The electrode 10 may however also have a different shape (for instance a rectangular or oval shape), as long as its functionality is preserved and the convenience for the user is sufficient.

The electrode 10 may be a dry electrode comprising a plurality of flexible electrode pins 14 (or electrode fingers 14) extending from the base portion 13 and configured to be placed on a subject's body. In this configuration, the electrode pins 14 or electrode fingers 14 may provide a direct measurement, without requiring the use of any intermediate contacting gel. In an alternative embodiment (not shown in the figures), the electrode may be a wet electrode, which may require the use of a contacting gel to provide a measurement. In the preferred embodiment, the electrode is a dry electrode. A dry electrode may have the convenience that no contacting gel needs to be used and that less cleaning is required after the use of the electrode.

The electrode 10 may be an injection-molded, electrically conductive element, as is known to the person skilled in the art. For example, the electrode 10 is made from a polymer doped with or comprising silver and/or silver chloride. However, the electrode 10 may also be manufactured via a different manufacturing method or be made from a different material or mix of materials, without departing from the scope of the present disclosure. The electrode 10 may be configured to obtain an electroencephalographic (EEG) measurement.

The electrode receptacle 20 may comprise an upper housing 24 and a lower housing 26. The upper housing 24 and the lower housing 26 may be attached to one another for example via a snap-fit coupling or a click-on coupling. The lower housing 26 may comprise a male snap feature 27 and the upper housing 24 may comprise a female snap feature 28, wherein the male and female snap features 27, 28 are configured to provide the snap-fit coupling. For instance, as shown in FIG. 1, the lower housing 26 may comprise a plurality of prongs 27 or hooks 27 configured to couple to respective holes 28 in the upper housing 24.

The upper housing 24 may also comprise a through-hole 33 for insertion of electronic wiring (not shown). In this configuration, only a minimal amount of wiring (for instance originating from a first circuit board, e.g. printed circuit board, PCB) will be exposed, since the majority of the wiring can be hidden in the cap including the electrode assembly 1, while only a minor portion of the wiring can be inserted through the through-hole 33 for contacting the electrode. This may allow convenient contacting of the electrode 10 to a first circuit board (not shown). This configuration also has the benefit that if the electrode is decoupled from the electrode receptacle 20, only a very limited amount of wiring will be exposed to the outside, thus reducing the risk of intended or unintended manipulation. In addition, the wiring can be fixed to an electronic circuit board 42 mounted to the upper housing 24, as shown in FIG. 1 and as will be further discussed below. In this configuration, practically no wiring will be exposed to the outside, even if the electrode 10 is decoupled from the electrode receptacle 20.

In one embodiment, the upper housing 24 may comprise a plurality of spacers 31 and the lower housing may comprise a plurality of spacers 29. The spacers 29, 31 may be arranged to face the respective other of the upper or lower housing 24, 26. The assembled upper and lower housing 24, 26 may form a housing for receiving and holding the electrode 10. Such spacers may enable or ensure the provision of a recess 35 between the upper and lower housing, as will be further discussed below. It is noted that the electrode receptacle 20 may also be provided as a unitary structure, without having two separate housing components. For instance, the electrode receptacle 20 may be injection-molded as one piece, including all the features discussed for the upper and lower housing 24, 26, albeit being present in the single structure. However, in this case, no snap-fit coupling may be required.

The assembly 1 comprises a coupling mechanism 30 configured to couple the electrode 10 to the electrode receptacle 20, the coupling mechanism 30 having a locked configuration, in which the electrode 10 is coupled to the electrode receptacle 20, and an unlocked configuration, in which the electrode 10 is decoupled from the electrode receptacle 20.

The coupling mechanism 30 may comprise a male coupling feature 32 and a female coupling feature 34. In the following, an embodiment will be described, in which the electrode 10 comprises the male coupling feature 32 and the electrode receptacle 20 comprises the female coupling feature 34. However, in an alternative embodiment, the electrode receptacle 20 may comprise the male coupling feature 32 and the electrode 10 may comprise the female coupling feature 34, without departing from the scope of the present disclosure. For instance, in FIG. 1, instead of a protrusion 32a, the electrode 10 may comprise at least one recess formed in the lateral surface 13 and the electrode receptacle 20 may comprise at least one protrusion (e.g., a pin) extending radially outwards from the inner surface 21. Each feature described herein may be combined with either of both alternative embodiments.

In one embodiment, as shown in FIG. 1, the electrode 10 comprises the male coupling feature 32 and the electrode receptacle 20 comprises the female coupling feature 34. The electrode 10 may comprise an essentially cylindrical base portion 12 having a lateral surface 13. As indicated above, the electrode 10 and its base portion 12 may also have a different shape having a lateral (e.g., outside) surface 13. The coupling mechanism 30 may comprise at least one (e.g., three) protrusion 32a forming a male coupling feature 32. The at least one protrusion 32a may extend radially outwards from the lateral surface 13 and may extend across only a part of a circumference of the lateral surface 13 of the base portion 12 of the electrode 10.

The electrode receptacle 20 may comprise an opening 23 for receiving the electrode 10, the opening 23 having an inner surface 21. The opening 23 may be an essentially circular opening 23 having a radial inner surface 21. In a preferred embodiment, the electrode 10 has an essentially cylindrical shape and the opening 23, which is configured to receive the electrode 10, is an essentially circular opening 23 having a radial inner surface 21. It is however noted that the opening may also be for instance rectangular, while still allowing an essentially cylindrically shaped electrode to be coupled in the way described herein. The inner surface 21 may be part of or formed by the lower housing 26. The electrode receptacle 20 may comprise at least one (e.g., three) second protrusion 34a extending radially inwards from the inner surface 21 at the opening 23. The at least one second protrusion 34a may extend across only a part of a circumference of the inner surface 21. The upper housing 24 may be configured to at least partially cover the opening 23, for instance with an inner wall 25.

At least one (e.g., three) recess 35 may be axially confined by the at least one second protrusion 34a. The at least one recess 35 may be formed between the at least one second protrusion 34a and an inner wall 25 of the upper housing 24 of the electrode receptacle 20. Spacers 29, 31 (already discussed above) may be used to ensure that the recess 35 has a predefined axial height, i.e., define a lower limit of a distance between the at least one second protrusion 34a and the inner wall 25. An upper limit of the distance between the at least one second protrusion and 34a and the inner wall 25 may be defined by the male and female snap-fit coupling features 27, 28 discussed above. The at least one recess 35 may be configured to engage the at least one protrusion 32a. It is again emphasized that all the elements described herein may be present on a unitary electrode receptacle 20, consisting of a unitary housing formed as one piece.

In one embodiment, the coupling mechanism 30 may be a turn-lock assembly that works similar to a bayonet mount. The coupling mechanism 30 may be configured to be shifted between the unlocked configuration and the locked configuration by manually turning or rotating the electrode 10 relative to the electrode receptacle 20. In this configuration, the at least one protrusion 32a, in the locked configuration of the coupling mechanism 30, may be sandwiched between the at least one second protrusion 34a and the inner wall 25. This may block movement of the electrode 10 relative to the electrode receptacle 20 in the axial direction (e.g., the direction along the center axis 17 or rotation axis 17 of the base portion 12 of the electrode 10). It is again emphasized that all the male and female coupling features described herein may be inversed, i.e., the electrode 10 may comprise the female coupling feature and the electrode assembly 20 may comprise the male coupling feature.

The base portion 12 of the electrode 10 may have an upper axial end surface 15 facing an inner wall 25 of the electrode receptacle 20, wherein said axial surface 15 comprises at least one (e.g., three) groove 36. The at least one groove 36 may be formed as a circle segment around axis 17 and parallel to the lateral surface 13. The at least one groove 36 may have a linearly varying depth along its length, thereby forming a ramp-like structure. The at least one groove 36 may be configured to couple with at least one pin 38 extending axially from the inner wall 25. The at least one pin 38 may be fixed to the inner wall 25 via a socket 39. In an alternative configuration, the electrode 10 may comprise at least one pin 38 extending axially from the upper axial end surface 15 and the electrode receptacle 20 may comprise at least one groove 36 formed at the inner wall 25.

The at least one pin 38 and the at least one groove 36 may be arranged such that, when the electrode 10 is rotated relative to the electrode receptacle 20 to shift the coupling mechanism 30 between the locked configuration and the unlocked configuration, the at least one pin 38 slides along the at least one groove 36. The at least one pin 38 may be configured to extend into the at least one groove 36 and contact a bottom surface of the at least one groove 36. As the bottom surface ascends along the length of the at least one groove 36, the at least one pin 38 and, thus, the electrode 10, may be translationally moved in an axial direction relative to the electrode receptacle 20 when shifting the coupling mechanism 30 between the unlocked configuration and the locked configuration. Thus, the coupling mechanism 30 may be shifted between the unlocked configuration and the locked configuration by both rotating and axially moving the electrode 10 relative to the receptacle 20. The at least one pin 38 and the at least one groove 36 may therefore be used as a “guiding mechanism” facilitating coupling between the electrode 10 and the receptacle 20.

It is noted that the at least one groove 36, the at least one pin 38 and the male and female coupling features 32, 34 may be formed such that the coupling mechanism 30 is shifted between the unlocked configuration and the locked configuration by only translationally moving the electrode 10 relative to the electrode receptacle 20 (i.e., without rotating the electrode relative to the electrode receptacle 20). Such a shifting of the coupling mechanism 30 may also be obtained without providing the at least one groove 36 and the at least one pin 38. In this case, for example, the electrode may be press-fit or snap-fit into the electrode receptacle by inserting it in an axial direction, thereby shifting the coupling mechanism into the locked configuration.

Alternatively, the at least one groove 36, the at least one pin 38 and the male and female coupling features 32, 34 may be formed such that the coupling mechanism 30 is shifted between the unlocked configuration and the locked configuration without translationally moving the electrode 10 relative to the electrode receptacle 20. In this case, for example, the at least one pin 38 and/or the bottom surface of the at least one groove 36 may be deformed when rotating the electrode 10 relative to the electrode receptacle 20, or the at least one pin 38 may not contact the bottom surface of the at least one groove 36. As another example, the groove 36 may have a constant depth along the length of the grove 36, or no groove 36 and pin 38 may be provided.

The axial surface 15 of the base portion 12 may comprise at least one blocking cavity 40 configured to couple with the at least one pin 38. As can be seen in FIG. 1, the at least one blocking cavity 40 may be arranged at a longitudinal end of the at least one groove 36. Thus, when the coupling mechanism 30 is shifted from the unlocked configuration to the locked configuration, the pin 38 slides along the at least one groove 36. The at least one pin 38 and/or the bottom surface of the at least one groove 36 may be deformed when rotating the electrode 10 relative to the electrode receptacle 20, as noted above. Once the coupling mechanism reaches the locked configuration, the at least one pin 38 may snap into the at least one blocking cavity 40. This configuration may avoid the electrode 10 from turning or decoupling unintentionally. That is, the combination of the at least one pin 38 and the at least one blocking cavity may provide a “retaining mechanism” for retaining a relative (e.g., rotational) position between the electrode 10 and the receptacle 20. Thus, in the locked configuration, the pin may extend into the blocking cavity 40 such that it hinders rotation of the electrode 10 relative to the receptacle 20. A predefined torque may be required to rotate the electrode 10 such that the coupling mechanism 30 is shifted from the locked configuration to the unlocked configuration. The predefined torque may depend on the dimensions of the pin 38, the blocking cavity 40, and the material of the electrode 10 and the pin 38. The assembly 1 may be configured such that the predefined torque is around 1 to 10 Nm, for example between 1 and 1.2 Nm. The skilled person will know, based on the information provided herein and based on practicability for the user, how to provide a torque useful for the purpose of locking and unlocking the coupling mechanism as described above.

The axial surface 15 of the base portion 12 of the electrode 10 may comprise at least one trench 16 configured to receive electronic wiring or an electrode receptacle contact. This electronic wiring may originate from a first printed circuit board (already described above), and may have been inserted through a through-hole 33 in the upper housing 24. Said axial surface 15 may comprise an electrode contact region (e.g., in the trench 16) configured to contact an electrode receptacle contact. The electrode assembly 1 may further comprise an electrical pressure contact element 41 and an electronic circuit board 42. The electronic circuit board 42 may be clip-mounted, glued or otherwise attached to the upper housing 24, wherein the electrical pressure contact element 41 may be coupled to the electronic circuit board 42. That is, both the electronic circuit board 42 and electrical pressure contact element 41 may be arranged in between the upper housing 24 and the lower housing 26. In a unitary housing configuration, both the electronic circuit board 42 electrical pressure contact element 41 may be arranged within the unitary housing. The electrode receptacle contact may be the electrical pressure contact element 41. The electrical pressure contact element 41 may be or comprise a spring contact exerting a contact pressure onto the electrode contact region of the electrode 10, when the electrode 10 is being held by the receptacle 20. The spring contact may be made from or plated with an electrically conductive material, for example gold. The electrically conductive material may be selected such that no corrosion occurs at the electrode 10 and/or the spring contact (e.g., when the electrode 10 is in contact with the spring contact) and may for instance be selected from inert metals such as gold described above. Thereby, an electrical connection between the electrode 10 and the electronic circuit board 42 may be ensured whilst using an electrode 10 without wiring or a wiring interface. This configuration may have the benefit that the electrode 10 can be readily coupled to and decoupled from the electrode receptacle 20 without having to consider any wiring or electrical contact. As an additional benefit, less or no wiring will be exposed to the outside, such that the assembly will be less prone to manipulation and failure.

The assembly 1 may further comprise wiring (not shown) electrically connected to the electronic circuit board 42. As described above, the upper housing 24 may comprise a through-hole 33 through which the wiring may extend from an exterior of the electrode receptacle 20 to the electronic circuit board 42. The through-hole 33 may further be configured to hold the electronic circuit board 42 in place relative to the upper housing 26. The wiring may connect the electronic circuit board 42 to the first circuit board described herein.

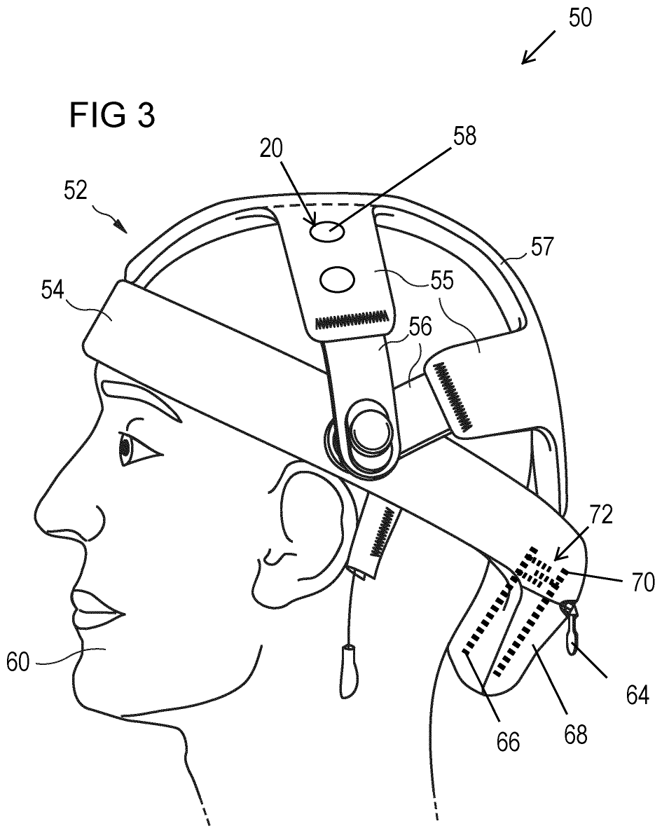

FIG. 3 shows a system 50 comprising a holder 52 or headset 52 configured to hold the electrode receptacle 20 in a fixed position relative to the surface of a subject's body. The holder 52 may comprise a plurality of bands or straps 54, 55, 57. A middle band 57 may be configured to extend in a sagittal plane of the user. A headband 54 may be configured to encircle the user's head 60. One or more sidebands 55 may be connected to both the middle band 57 and the headband 54, for example via connecting portions 56. The system 50 may also comprise the receptacle 20, which is embedded in one of the bands 54, 55, 57. In the illustration of FIG. 3, an upper surface 58 of the upper housing 24 can be seen, whereas the lower housing 26 and the electrode 10 are covered by the side band 55.

The spacers 29, 31 may act as teeth for fixing the electrode receptacle onto the band 54. Namely, the band 54 may comprise a through-hole (not shown) in which the recess 35 is located, wherein a portion of the band 54 adjacent to the through-hole is sandwiched between the spacers 29, 31 of the upper housing 24 and the lower housing 26. In an alternative configuration, the entire housing may be covered by the band 54. In this configuration, the band 54 may comprise an inner portion configured to be sandwiched between spacers 29, 31 of the upper housing 24 and the lower housing 26, and an outer portion configured to cover the assembly 1. The holder 52 in the shown example is a headset or cap configured to hold the receptacle 20 in one of the positions of the standardized 10-10 or 10-20 EEG placement scheme on a head 60 of a user.

The holder 52 may comprise the first circuit board mentioned above, as indicated in FIG. 3 with reference sign 66. The first circuit board 66 may be arranged at an element or portion of the holder 52 to be placed on a rear side of the user's head 60. The wiring mentioned above may electrically connect the first circuit board 66 with the electronic circuit board 42 and/or the electrical pressure contact element 41.

The first circuit board 66 may be arranged within a closable pocket 68 attached to or formed by (e.g., a headband of) the headset 52. The closable pocket 68 may be configured to be arranged at a rear side of the user's head 60 when the user is wearing the headset 52. The pocket 68 may be closable via a zip fastener 64.

The aforementioned wiring may be attached (e.g., glued or stitched) to or embedded in the headband 54, the middle band 57 and/or the at least one sideband 55 of the headset 52. The wiring may extend in a zig-zag pattern or a wave pattern. The wiring may comprise electrically shielded wires. The wiring may be covered with a (e.g., electrically shielding) protective cover.

The headset 52 may further comprise a second circuit board 70 comprising circuitry for collecting an electroencephalography, EEG, measurement signal from or via the first circuit board 66. The second circuit board 70 may be arranged at the element or portion of the headset 52 to be placed on the rear side of the user's (e.g., patient's or wearer's) head 60. The second circuit board 70 may be removably coupled to the first circuit board 66. The second circuit board 70 may be removably coupled to the first circuit board 66 via an (e.g., push-in) electrical connector 72. The electrical connector 72 may be a board-to-board connector or a first circuit board, PCB, connector. The second circuit 70 board may be arranged in the closable pocket 68 together with the first circuit board 66. The first circuit board 66 and the second circuit board 70 may each be a printed circuit board, a flexible circuit board, an etched circuit board or a circuit board comprising soldered wiring instead of printed conductive traces.

The coupling mechanism may be configured to be shifted between the unlocked configuration and the locked configuration (e.g., only) by assembling or disassembling (e.g., coupling or de-coupling) the upper housing 24 and the lower housing 26. In this case, the upper housing 24 and the lower housing 26 may be configured to hold the electrode 10 in place (e.g., in a fixed spatial relationship relative to the electrode receptacle 20) when being assembled (e.g., when being in an assembled state) and may be configured to release the electrode 10 when being disassembled (e.g., when being in a disassembled state). For instance, in this configuration, the electrode receptacle 20 may have an opening 23 and an inner lateral surface 21 comprising the at least one second protrusion 34a. The at least one second protrusion 34a may be configured to lock the electrode 10 in position after the lower housing 26 has been assembled with the upper housing 24. In this configuration, a turning or rotating of the electrode 10 relative to the electrode receptacle 20 and/or a translational movement of the electrode 10 relative to the electrode receptacle 20 may not allow the decoupling of the electrode 10 from the electrode receptacle 20. Instead, the electrode 10 may only be removable, if the lower housing 26 is disassembled from the upper housing 24. Thus, the electrode 10 may be exclusively held within the electrode receptacle 20 by at least one protrusion (e.g., the at least one second protrusion 34a) of the lower housing 26 at least partially covering the electrode 10 (e.g., axially covering the protrusion 32). In this configuration, the electrode 10 may only be released if the lower housing 26 is disassembled from the upper housing 24. Alternatively, the electrode receptacle 20 may be configured such that a turning or rotating of the electrode 10 relative to the electrode receptacle 20 and/or a translational movement of the electrode 10 relative to the electrode receptacle 20 also releases the electrode 10 from the receptacle 20.

It is noted that the individual components, elements, embodiments and examples described above may be used in any combination. For instance the coupling mechanisms 30 described herein may be combined with one another. In other words, the coupling mechanism 30 of the electrode assembly 1 may be configured to be shifted between the locked configuration and the unlocked configuration when rotating the electrode 10 relative to the receptacle 20, when transversally moving the electrode 10 relative to the receptacle 20 and/or or when assembling or disassembling the upper and lower housing 24, 26. The coupling mechanism 30 may be used in combination with an electrode receptacle consisting of an upper and a lower housing. In case the coupling mechanism 30 is not configured to be shifted between the locked configuration and the unlocked configuration when assembling or disassembling the upper and lower housing 24, 26, it may be used with an electrode receptacle being formed of a unitary structure. Further, the coupling mechanism 30 may be combined with at least one of a guiding mechanism (e.g., as described above), a retaining mechanism (e.g., as described above), the electrical pressure contact element 41 and/or the electronic circuit board 42, the trench 16, the through-hole 33, etc. For instance, the coupling mechanism 30 may be used in combination with the retaining mechanism, without the guiding mechanism. Alternatively, the coupling mechanism 30 may be used in combination with the guiding mechanism, without the retaining mechanism. In some embodiments, the electronic circuit board 42 may be positioned outside of the assembly 1 and thus not be part of the assembly 1 itself. The advantages of each feature and the combinations will become apparent to the skilled person based on the disclosure herein.

As will be apparent from the above, the present disclosure provides for an advantageous technique for holding an electrode. Namely, the electrode 10 may be removably held by, or coupled to, the electrode receptacle 20. This may enable easily replacing the electrode 10. Whilst the groove 36 may assist in aligning the electrode 10 relative to the receptacle 20 during shifting of the coupling mechanism 30 from the unlocked configuration to the locked configuration, the blocking cavity 40 may ensure that the electrode 10 is not unintentionally decoupled from the receptacle 20. The electrical pressure contact element 41, the electronic circuit board 42 and the axial surface of the electrode 10 may provide a reliable electrical connection with low assembly effort.

The electrode assembly described herein may provide for an easy exchange or replacement of electrodes. The described techniques may facilitate the replacement of an electrode if a different type or size of electrode is required for a particular measurement. Further, the described techniques may facilitate the replacement of an electrode if a specific electrode is defective. In traditional systems, the cap may no longer be usable, if one individual electrode is malfunctioning, thus requiring a cumbersome replacement of the electrode or the entire disposal of the cap. Further, the described techniques may facilitate the cleaning of the electrodes, without having to clean the entire system including the cap. It is noted in this connection that caps may include soft material (such as polyurethane) to increase the convenience and comfort for the user, wherein such soft materials may soak up any cleaning solution used for cleaning the electrodes. Since with the described techniques individual electrodes can be decoupled and cleaned individually, no cumbersome cleaning of the entire cap is required.

Alternative and/or additional advantages may be obtained with the subject matter disclosed herein.

1. An electrode assembly, comprising:
an electrode configured to be placed on a surface of a subject's body for recording an electric body signal;
an electrode receptacle configured to receive the electrode; and
a coupling component configured to couple the electrode to the electrode receptacle, the coupling component having a locked configuration, in which the electrode is coupled to the electrode receptacle, and an unlocked configuration, in which the electrode is decoupled from the electrode receptacle.

2. The electrode assembly of claim 1, wherein the coupling component comprises at least one of a bayonet mount assembly, a bayonet connector assembly, a turn-lock assembly, a screw assembly, a snap-fit assembly, a friction assembly, or a magnetic assembly,
wherein the coupling component is configured to be shifted between the unlocked configuration and the locked configuration by turning or rotating the electrode relative to the electrode receptacle.

3. The electrode assembly of claim 1, wherein the coupling component is configured to be shifted between the unlocked configuration and the locked configuration by translationally moving the electrode relative to the electrode receptacle.

4. The electrode assembly of claim 1,
wherein the coupling component comprises a male coupling feature and a female coupling feature,
wherein the electrode receptacle comprises the male coupling feature and the electrode comprises the female coupling feature, or wherein the electrode comprises the male coupling feature and the electrode receptacle comprises the female coupling feature.

5. The electrode assembly of claim 1,
wherein the coupling component comprises at least one protrusion,
wherein at least one of the electrode or the electrode receptacle comprises a base portion having a lateral surface,
wherein the at least one protrusion extends radially from the lateral surface, and
wherein the coupling component comprises at least one recess at the respective other of the electrode or electrode receptacle, the at least one recess being configured to engage the at least one protrusion.

6. The electrode assembly of claim 5,
wherein the electrode comprises the base portion having the lateral surface from which the at least one protrusion extends radially outwards,
wherein the at least one protrusion extends across at least a part of a circumference of the lateral surface, and
wherein the at least one recess is formed at the electrode receptacle.

7. The electrode assembly of claim 6,
wherein the electrode receptacle comprises an opening for receiving the electrode, the opening having an inner surface,
wherein the electrode receptacle comprises at least one second protrusion extending radially inwards from the inner surface, and
wherein the at least one recess is axially confined by the at least one second protrusion and wherein the recess is formed by the at least one second protrusion and an inner wall of the electrode receptacle.

8. The electrode assembly of claim 5,
wherein the base portion of the electrode or the electrode receptacle has an axial surface facing a surface of the respective other of the electrode or electrode receptacle,
wherein said axial surface comprises at least one groove,
wherein the at least one groove has a varying depth along its length, and
wherein the at least one groove is configured to couple with at least one pin arranged at the surface of the respective other of the electrode or the electrode receptacle.

9. The electrode assembly of claim 8,
wherein the at least one pin is arranged such that it extends into the at least one groove when the coupling component is shifted between the locked configuration and the unlocked configuration,
wherein the at least one pin and the at least one groove are arranged such that, when the coupling component is shifted between the locked configuration and the unlocked configuration, the at least one pin is moved along the at least one groove.

10. The electrode assembly of claim 6,
wherein the base portion of the electrode or the electrode receptacle has an axial surface facing a surface of the respective other of the electrode or electrode receptacle,
wherein said axial surface comprises at least one blocking cavity,
wherein the at least one blocking cavity is configured to couple with at least one pin arranged at the surface of the respective other of the electrode or the electrode receptacle.

11. The electrode assembly of claim 10,
wherein the at least one pin and the at least one blocking cavity are arranged such that the at least one pin extends into the at least one blocking cavity when the coupling component is in the locked configuration, and does not extend into the at least one blocking cavity when the coupling component is in the unlocked configuration.

12. The electrode assembly of claim 11,
wherein the at least one blocking cavity is arranged at a longitudinal end of the at least one groove,
wherein the at least one pin, the at least one groove and the at least one blocking cavity are arranged such that, when the coupling component is shifted from the unlocked configuration to the locked configuration, the at least one pin is moved along the at least one groove, and once the coupling
component reaches the locked configuration, the at least one pin extends into the at least one blocking cavity.

13. The electrode assembly of claim 1,
wherein the electrode receptacle comprises an upper housing and a lower housing, wherein the upper housing and the lower housing are configured to be coupled to one another to form a housing for receiving the electrode, and
wherein at least one male coupling feature or female coupling feature is formed by the coupled upper housing and lower housing.

14. The electrode assembly of claim 13,
wherein the coupling component is configured to be shifted between the unlocked configuration and the locked configuration by assembling or disassembling the upper housing and the lower housing,
wherein the upper housing and the lower housing are configured to hold the electrode in place when being assembled and are configured to release the electrode when being disassembled.

15. The electrode assembly of claim 7,
wherein the electrode receptacle comprises an upper housing and a lower housing,
wherein the upper housing and the lower housing are configured to be coupled to one another to form a housing for receiving the electrode, and
wherein at least one male coupling feature or female coupling feature is formed by the coupled upper housing and lower housing,
wherein the lower housing comprises the at least one second protrusion and the upper housing is configured to at least partially cover the opening,
wherein the recess is formed by the at least one second protrusion and an inner wall of the upper housing.

16. The electrode assembly of claim 1,
wherein the base portion of the electrode has an axial surface facing a surface of the electrode receptacle,
wherein said axial surface comprises at least one trench configured to receive electronic wiring or an electrode receptacle contact and/or wherein said axial surface comprises an electrode contact region configured to contact an electrode receptacle contact.

17. The electrode assembly of claim 1, further comprising:
an electrical pressure contact element arranged in a fixed pose relative to the electrode receptacle and configured to contact the electrode when the coupling component is in the locked configuration,
wherein the electrode assembly further comprises an electronic circuit board arranged on the electrode receptacle, wherein the electrical pressure contact element is arranged on the electronic circuit board, wherein further the electrode receptacle contact is the electrical pressure contact element.

18. An electrode for recording an electric body signal comprising:
a base portion;
a plurality of electrode pins attached to the base portion and configured to be placed on a surface of a subject's body for recording an electric body signal; and
at least one coupling feature for coupling to an electrode receptacle, wherein the at least one coupling feature extends radially with respect to the base portion, and wherein the at least one coupling feature extends across at least a part of a circumference of the base portion.

19. An electrode receptacle for an electrode for recording an electric body signal, the electrode receptacle comprising:
an opening for receiving the electrode, wherein the electrode is configured to be placed on a surface of a subject's body for recording an electric body signal;
at least one coupling feature for coupling to the electrode, wherein the at least one coupling feature extends radially with respect to the opening and extends across at least a part of a circumference of the opening; and
an electrical pressure contact element arranged in a fixed position relative to the electrode receptacle and configured to contact the electrode when the electrode is held in a fixed position relative to the receptacle by the at least one coupling feature.

20. A system comprising:
the assembly according to claim 1, or the electrode receptacle according to claim 19; and
a holder configured to hold the receptacle in a fixed position relative to the surface of a subject's body.
